(19) United States
(12) Patent Application Publication
(10) Pub. No.: US 2025/0241702 A1
(43) Pub. Date: Jul. 31, 2025

(54) METHOD AND APPARATUS FOR SUPPLYING MONOPOLAR AND BIPOLAR CURRENT BY SKIN CONTACT AND SKIN DEPTH

(71) Applicant: SKINGRAB CO., LTD., Seoul (KR)

(72) Inventor: Sugun LEE, Paju-si (KR)

(73) Assignee: SKINGRAB CO., LTD., Seoul (KR)

(21) Appl. No.: 19/060,649

(22) Filed: Feb. 22, 2025

Related U.S. Application Data
(63) Continuation of application No. PCT/KR2022/020015, filed on Dec. 9, 2022.

(30) Foreign Application Priority Data
Aug. 22, 2022 (KR) 10-2022-0104506

(51) Int. Cl. A61B 18/14; A61B 17/00; A61B 18/00; A61B 18/12; A61B 18/16

(52) U.S. Cl. CPC A61B 18/1477; A61B 2017/0046; A61B 2018/0016; A61B 2018/0047; A61B 2018/00702; A61B 2018/0072; A61B 2018/00761; A61B 2018/00875; A61B 2018/1253; A61B 2018/126; A61B 2018/167

(57) ABSTRACT

The present disclosure may include an electrode module including a plurality of first electrodes of a needle type inserted into a skin and a plurality of second electrodes of a contact type contacting a surface of the skin; an energy supply module configured to apply a first current of a monopolar type to the first electrode and a second current of a bipolar type to the second electrode; a transfer module configured to move the electrode module so that the first electrode reaches a target depth within the skin and the second electrode contacts the surface of the skin; and a controller configured to control the energy supply module so that the first current and the second current are alternately applied based on the first electrode reaching the target depth and the second electrode contacting the surface of the skin, wherein the first electrode and the second electrode are provided in a cartridge housing coupled to a handpiece, and wherein the first electrode is provided in a central portion of the cartridge housing, and the second electrode is provided in a periphery of the first electrode.

### CROSS-REFERENCE TO RELATED APPLICATIONS

The present application is a continuation of International Patent Application No. PCT/KR2022/020015, filed on Dec. 9, 2022, which is based upon and claims the benefit of priority to Korean Patent Application No. 10-2022-0104506 filed on Aug. 22, 2022. The disclosures of the above-listed applications are hereby incorporated by reference herein in their entirety.

### TECHNICAL FIELD

The present disclosure relates to a method and apparatus for supplying monopolar and bipolar current. More particularly, the present disclosure relates to a method and apparatus for supplying monopolar and bipolar current by skin contact and skin depth.

### BACKGROUND ART

Generally, skin care devices for wrinkle removal, skin elasticity recovery, and sebum removal include methods such as delivering ultrasound to skin tissue (HIFU type), delivering high-frequency to skin tissue (RF type), and irradiating laser light to skin tissue (Optical type).

Devices that deliver high-frequency waves to skin tissue repeatedly infiltrate deep skin (e.g., dermal layer) of the skin (e.g., face) with RF needle electrodes that move back and forth in a vertical direction, and use the heat generated by high-frequency to remove damaged collagen, elastic fibers, and the like from the deep skin of the target point and promote new formation.

Furthermore, these skin care devices improve skin pigmentation, acne scars, and wrinkles.

That is, after applying various energies to the target area of the skin to intentionally cause wounds, they stimulate collagen in the dermal layer to induce collagen regeneration, thereby regenerating the skin area.

However, the conventional skin care devices are not able to deliver abundant electric energy or efficient electric energy to a specific depth of the skin.

Therefore, the conventional skin care devices are not able to accurately irradiate abundant electric energy or efficient electric energy during treatment, resulting in poor treatment accuracy.

In addition, the conventional skin care devices have limitations in shortening treatment time and maximizing treatment effects because doctors have to perform the treatment carefully.

### DETAILED DESCRIPTION OF THE INVENTION

### Technical Problem

The embodiment of the present disclosure is to accurately investigate abundant electric energy or efficient electric energy during a procedure, thereby increasing the accuracy of the procedure.

In addition, the embodiment of the present disclosure is to shorten a procedure time and maximize the procedure effect.

In addition, the embodiment of the present disclosure is to maximize an optimal skin improvement effect of a monopolar type and a bipolar type while preventing an occurrence of skin burns in advance.

In addition, the embodiment of the present disclosure is to efficiently increase a procedure effect by changing the range of skin tissue that can be stimulated depending on a low-frequency current and a high-frequency current.

Technical problems of the inventive concept are not limited to the technical problems mentioned above, and other technical problems not mentioned will be clearly understood by those skilled in the art from the following description.

### Technical Solution

In an aspect of the present disclosure, an apparatus for supplying monopolar and bipolar current by skin contact and skin depth may include an electrode module including a plurality of first electrodes of a needle type inserted into a skin and a plurality of second electrodes of a contact type contacting a surface of the skin; an energy supply module configured to apply a first current of a monopolar type to the first electrode and a second current of a bipolar type to the second electrode; a transfer module configured to move the electrode module so that the first electrode reaches a target depth within the skin and the second electrode contacts the surface of the skin; and a controller configured to control the energy supply module so that the first current and the second current are alternately applied based on the first electrode reaching the target depth and the second electrode contacting the surface of the skin, wherein the first electrode and the second electrode are provided in a cartridge housing coupled to a handpiece, and wherein the first electrode is provided in a central portion of the cartridge housing, and the second electrode is provided in a periphery of the first electrode.

Furthermore, the controller may be configured to measure skin impedance based on the first electrode or the second electrode, and output energy corresponding to the measured skin impedance among preset energies, and further control the energy supply module so that the second electrode transmits energy corresponding to the skin impedance to the skin surface, and the first electrode transmits energy corresponding to the skin impedance when the target depth is reached.

Furthermore, the controller may be configured to control the energy supply module so that the first current is applied to the first electrode at a first current intensity corresponding to the reached target depth, and the second current is applied to the second electrode at a second current intensity corresponding to the surface of the contacted skin, wherein the first current intensity and the second current intensity are identical or different from each other.

Furthermore, the first current intensity and the second current intensity may be at least one of a low-frequency current or a high-frequency current.

Furthermore, the controller may be configured to control the energy supply module so that, based on the first electrode reaching the target depth and the second electrode contacting the surface of the skin, the first current and the second current are applied in a preset application order of the low-frequency current and the high-frequency current for a preset time.

Furthermore, the controller may be configured to control the energy supply module so that, based on the first electrode reaching the target depth, the first current is applied to either one of the low-frequency current and the high-frequency current for a preset time and for a preset target depth.

Furthermore, in another aspect of the present disclosure, a method performed by an apparatus for supplying monopolar and bipolar current by skin contact and skin depth may include moving, by a transfer module of the apparatus, an electrode module of the apparatus so that a first electrode reaches a target depth within the skin and a second electrode contacts a surface of the skin; determining, by a controller of the apparatus, whether the first electrode reaches the target depth and the second electrode contacts the surface of the skin; and controlling, by the controller of the apparatus, an energy supply module of the apparatus so that the first current and the second current are alternately applied based on the first electrode reaching the target depth and the second electrode contacting the surface of the skin, wherein the first electrode and the second electrode are provided in a cartridge housing coupled to a handpiece, and wherein the first electrode is provided in a central portion of the cartridge housing, and the second electrode is provided in a periphery of the first electrode.

Furthermore, controlling the energy supply module of the apparatus may include: measuring skin impedance based on the first electrode or the second electrode, and outputting energy corresponding to the measured skin impedance among preset energies, and controlling the energy supply module so that the second electrode transmits energy corresponding to the skin impedance to the skin surface, and the first electrode transmits energy corresponding to the skin impedance when the target depth is reached.

Furthermore, controlling the energy supply module of the apparatus may include: controlling, by the controller, the energy supply module so that the first current is applied to the first electrode at a first current intensity corresponding to the reached target depth, and the second current is applied to the second electrode at a second current intensity corresponding to the surface of the contacted skin, wherein the first current intensity and the second current intensity are identical or different from each other.

Furthermore, the first current intensity and the second current intensity may be at least one of a low-frequency current or a high-frequency current.

Furthermore, controlling the energy supply module of the apparatus may include controlling, by the controller, the energy supply module so that, based on the first electrode reaching the target depth and the second electrode contacting the surface of the skin, the first current and the second current are applied in a preset application order of the low-frequency current and the high-frequency current for a preset time.

Furthermore, controlling the energy supply module of the apparatus may include controlling, by the controller, the energy supply module so that, based on the first electrode reaching the target depth, the first current is applied to either one of the low-frequency current and the high-frequency current for a preset time and for a preset target depth.

Furthermore, in still another aspect of the present disclosure, a computer program stored in a computer-readable storage medium, when executed by one or more processors, may execute the following operations to perform a method performed by an apparatus for supplying monopolar and bipolar current by skin contact and skin depth, and the operations may include: an operation of moving, by a transfer module of the apparatus, an electrode module of the apparatus so that a first electrode reaches a target depth within the skin and a second electrode contacts a surface of the skin; an operation of determining, by a controller of the apparatus, whether the first electrode reaches the target depth and the second electrode contacts the surface of the skin; and an operation of controlling, by the controller of the apparatus, an energy supply module of the apparatus so that the first current and the second current are alternately applied based on the first electrode reaching the target depth and the second electrode contacting the surface of the skin, wherein the first electrode and the second electrode are provided in a cartridge housing coupled to a handpiece, and wherein the first electrode is provided in a central portion of the cartridge housing, and the second electrode is provided in a periphery of the first electrode.

Furthermore, the operation of controlling may include: measuring skin impedance based on the first electrode or the second electrode, and outputting energy corresponding to the measured skin impedance among preset energies, and controlling the energy supply module so that the second electrode transmits energy corresponding to the skin impedance to the skin surface, and the first electrode transmits energy corresponding to the skin impedance when the target depth is reached.

Furthermore, the operation of controlling may include: controlling, by the controller, the energy supply module so that the first current is applied to the first electrode at a first current intensity corresponding to the reached target depth, and the second current is applied to the second electrode at a second current intensity corresponding to the surface of the contacted skin, wherein the first current intensity and the second current intensity are identical or different from each other.

Furthermore, the first current intensity and the second current intensity may be at least one of a low-frequency current or a high-frequency current.

Furthermore, the operation of controlling may include controlling, by the controller, the energy supply module so that, based on the first electrode reaching the target depth and the second electrode contacting the surface of the skin, the first current and the second current are applied in a preset application order of the low-frequency current and the high-frequency current for a preset time.

Furthermore, the operation of controlling may include controlling, by the controller, the energy supply module so that, based on the first electrode reaching the target depth, the first current is applied to either one of the low-frequency current and the high-frequency current for a preset time and for a preset target depth.

### Advantageous Effects of the Invention

According to the present disclosure, when performing a procedure, it is provided an effect of accurately irradiating abundant electric energy or efficient electric energy, thereby increasing the accuracy of the procedure.

In addition, according to the present disclosure, it is provided an effect of shortening the procedure time and maximizing the procedure effect.

In addition, according to the present disclosure, it is provided an effect of improving the optimal skin condition of the monopolar type and the bipolar type while preventing the occurrence of skin burns in advance.

In addition, according to the present disclosure, the range of skin tissue that can be stimulated varies depending on the low-frequency current and the high-frequency current, thereby providing an effect of efficiently increasing the procedure effect.

The effects of the present disclosure are not limited to the effects mentioned above, and other effects not mentioned can be clearly understood by those skilled in the art from the description.

### BRIEF DESCRIPTION OF THE DRAWINGS

FIG. 1 is a diagram illustrating a configuration of an apparatus for supplying monopolar and bipolar current by skin contact and skin depth according to the present disclosure.

FIGS. 2 and 3 are diagram illustrating that the electrode module of FIG. 1 is provided in a cartridge housing coupled to a handpiece.

FIG. 4 is a diagram illustrating an example of a process in which a plurality of electrodes of a contact type of an electrode module contact a surface of a skin and a plurality of electrodes of a needle type invade an inside of the skin by a movement of the transfer module of FIG. 1.

FIG. 5 is a diagram illustrating a structure of a plurality of electrodes of a needle type arranged in the electrode module of FIG. 4.

FIG. 6 is a diagram illustrating a plurality of electrode structures of a contact type arranged in the electrode module of FIG. 4

FIG. 7 is a diagram illustrating an example of a process of applying a first current of a monopolar type to the plurality of electrodes of the needle type of FIG. 5.

FIG. 8 is a diagram illustrating an example of a process of applying a second current of a bipolar type to the plurality of electrodes of the contact type of FIG. 6.

FIG. 9 is a diagram illustrating an example of a process of storing target depth data, low-frequency current data, and high-frequency current data in the memory of FIG. 1.

FIG. 10 is a flowchart showing a method for supplying monopolar and bipolar current by skin contact and skin depth according to the present disclosure.

FIGS. 11 and 12 are diagrams illustrating an example of a process of controlling low-frequency current and high-frequency current to be sequentially applied to the plurality of first electrodes in the control step of FIG. 10.

FIGS. 13 and 14 are diagrams illustrating an example of a process of controlling the application of the corresponding low frequency current and the corresponding high frequency current to the plurality of first electrodes according to the target depth in the control step of FIG. 10.

### BEST MODE

In the drawings, the same reference numeral refers to the same element. This disclosure does not describe all elements of embodiments, and general contents in the technical field to which the present disclosure belongs or repeated contents of the embodiments will be omitted. The terms, such as “unit, module, member, and block” may be embodied as hardware or software, and a plurality of “units, modules, members, and blocks” may be implemented as one element, or a unit, a module, a member, or a block may include a plurality of elements.

Throughout this specification, when a part is referred to as being “connected” to another part, this includes “direct connection” and “indirect connection”, and the indirect connection may include connection via a wireless communication network. Furthermore, when a certain part “includes” a certain element, other elements are not excluded unless explicitly described otherwise, and other elements may in fact be included.

Furthermore, when a certain part “includes” a certain element, other elements are not excluded unless explicitly described otherwise, and other elements may in fact be included.

In the entire specification of the present disclosure, when any member is located “on” another member, this includes a case in which still another member is present between both members as well as a case in which one member is in contact with another member.

The terms “first,” “second,” and the like are just to distinguish an element from any other element, and elements are not limited by the terms.

The singular form of the elements may be understood into the plural form unless otherwise specifically stated in the context.

Identification codes in each operation are used not for describing the order of the operations but for convenience of description, and the operations may be implemented differently from the order described unless there is a specific order explicitly described in the context.

Hereinafter, operation principles and embodiments of the present disclosure will be described with reference to the accompanying drawings.

In the present specification, a controller of an apparatus for supplying monopolar and bipolar current by skin contact and skin depth according to the present disclosure includes various devices that may perform computational processing and provide results to a user. For example, the controller of the apparatus for supplying monopolar and bipolar current by skin contact and skin depth during needle insertion according to the present disclosure may include a computer, a server device, and a portable terminal, or may be in the form of one of them.

Here, the computer may include, for example, a notebook, desktop, laptop, tablet PC, slate PC, etc. equipped with a web browser.

A server device is a server that communicates with an external device to process information, and may include an application server, a computing server, a database server, a file server, a mail server, a proxy server, and a web server.

A portable terminal may include, for example, all kinds of handheld-based wireless communication devices such as PCS (Personal Communication System), GSM (Global System for Mobile communications), PDC (Personal Digital Cellular), PHS (Personal Handyphone System), PDA (Personal Digital Assistant), IMT (International Mobile Telecommunication)-2000, CDMA (Code Division Multiple Access)-2000, W-CDMA (W-Code Division Multiple Access), WiBro (Wireless Broadband Internet) terminals, smart phones, etc., and wearable devices such as watches, rings, bracelets, anklets, necklaces, glasses, contact lenses, or head-mounted devices (HM Ds).

The apparatus for supplying monopolar and bipolar current by skin contact and skin depth according to the present disclosure includes, in the case that a plurality of first electrodes of a needle type inserted into the skin and a plurality of second electrodes of a contact type that reaches a target depth and contact a surface of the skin contact the surface of the skin, a controller configured to control an energy supply module so that a first current of a monopolar type and a second current of a bipolar type are alternately applied to the first electrode and the second electrode. At this time, the first electrode and the second electrode may be provided in a cartridge housing coupled to a handpiece, and the first electrode may be provided in a central portion of the cartridge housing and the second electrode may be provided in a periphery of the first electrode.

The apparatus for supplying monopolar and bipolar current by skin contact and skin depth may accurately irradiate abundant electric energy or efficient electric energy during a procedure, thereby increasing the accuracy of the procedure. In addition, the apparatus for supplying monopolar and bipolar current by skin contact and skin depth may shorten the procedure time and maximize the effect of the procedure. In addition, the apparatus for supplying monopolar and bipolar current by skin contact and skin depth may prevent skin burns in advance while improving the optimal skin condition of the monopolar type and bipolar type.

Hereinafter, the apparatus for supplying monopolar and bipolar current by skin contact and skin depth will be examined in detail.

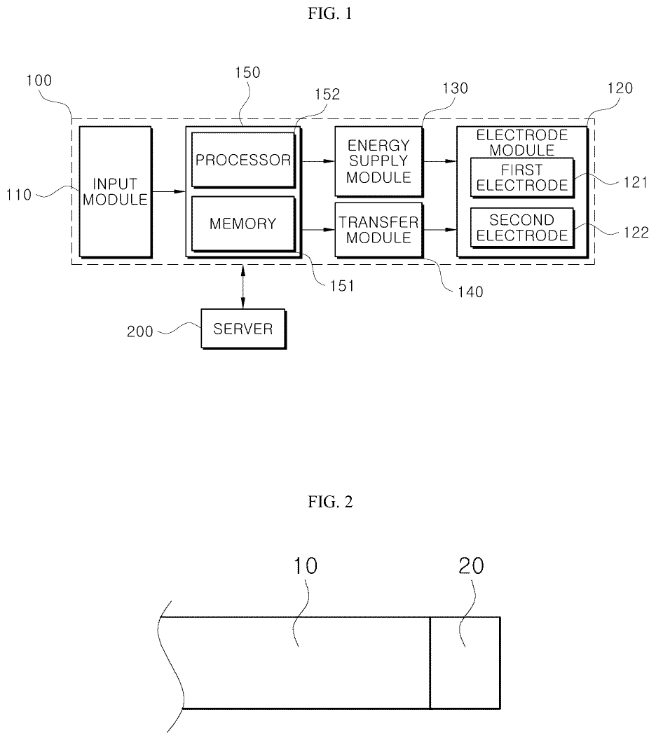

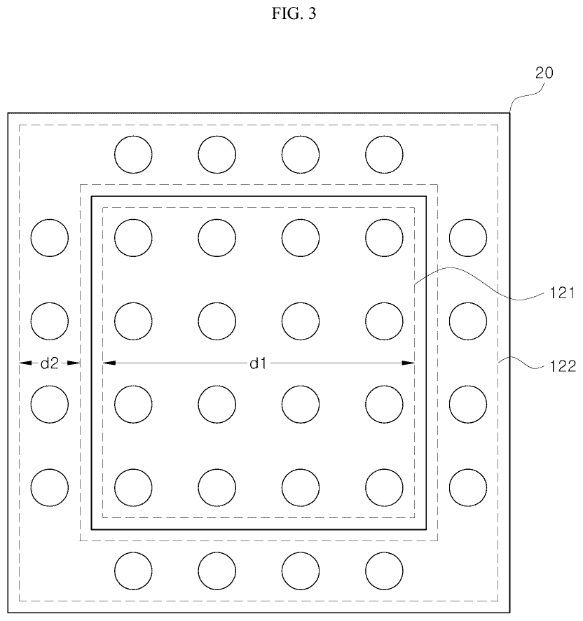

FIG. 1 is a diagram illustrating a configuration of an apparatus for supplying monopolar and bipolar current by skin contact and skin depth according to the present disclosure. FIGS. 2 and 3 are diagram illustrating that the electrode module of FIG. 1 is provided in a cartridge housing coupled to a handpiece.

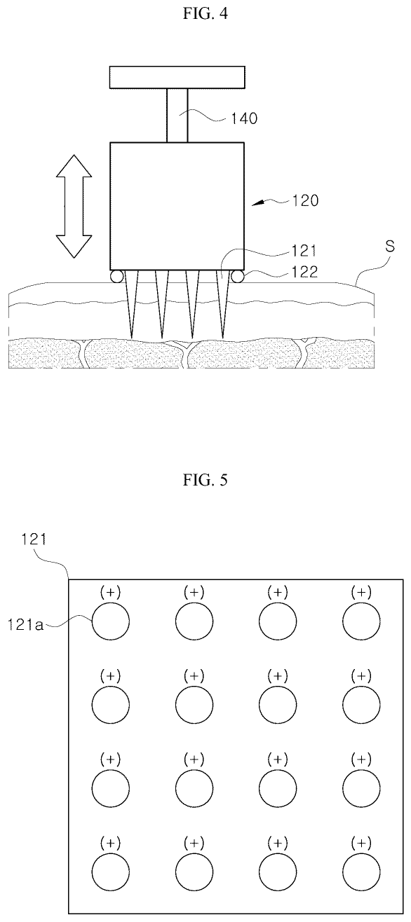

FIG. 4 is a diagram illustrating an example of a process in which a plurality of electrodes of a contact type of an electrode module contact a surface of a skin and a plurality of electrodes of a needle type invade an inside of the skin by a movement of the transfer module of FIG. 1.

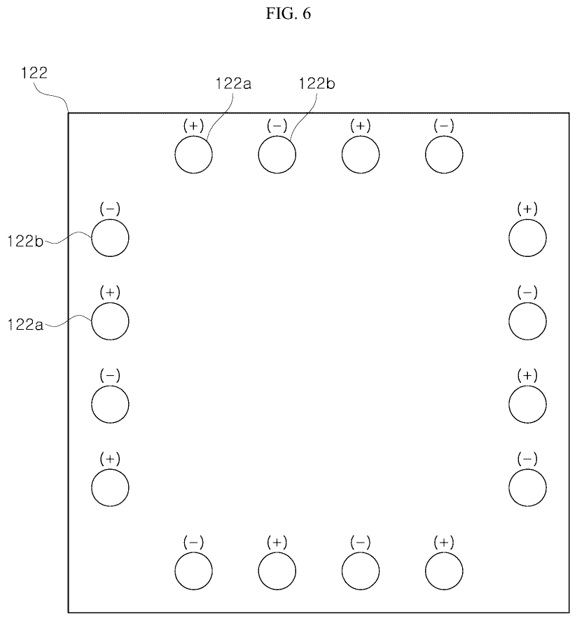

FIG. 5 is a diagram illustrating a structure of a plurality of electrodes of a needle type arranged in the electrode module of FIG. 4. FIG. 6 is a diagram illustrating a plurality of electrode structures of a contact type arranged in the electrode module of FIG. 4

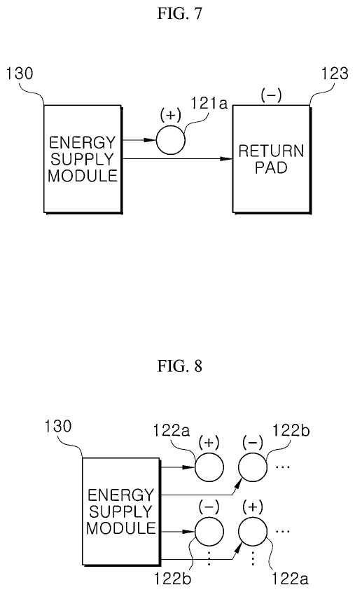

FIG. 7 is a diagram illustrating an example of a process of applying a first current of a monopolar type to the plurality of electrodes of the needle type of FIG. 5. FIG. 8 is a diagram illustrating an example of a process of applying a second current of a bipolar type to the plurality of electrodes of the contact type of FIG. 6.

Referring to FIGS. 1 to 8, an apparatus 100 for supplying monopolar and bipolar current by skin contact and skin depth may include an input module 110, an electrode module 120, an energy supply module 130, a transfer module 140, and a controller 150.

The input module 110 is configured to receive target depth information and treatment condition information within a skin from a user, and when the target depth information and the treatment condition information within the skin are input, the controller 150 may control an operation of the apparatus to correspond to the input target depth information and treatment condition information within the skin. Here, the target depth information within the skin may be a target depth value within a dermis layer, and the treatment condition information may be a treatment condition for removing wrinkles, a treatment condition for restoring skin elasticity, a treatment condition for removing sebum, and the like.

The input module 110 may include a hardware-type physical key (e.g., a button located on at least one of the front, rear, or side of the apparatus, a dome switch, a jog wheel, a jog switch, etc.) and a software-type touch key. As an example, a touch key may be formed as a virtual key, a soft key, or a visual key displayed on a touchscreen-type display module through software processing, or as a touch key placed on a part other than a touchscreen. Meanwhile, the virtual key or the visual key may be displayed on the touchscreen in various forms, and may be formed as, for example, a graphic, a text, an icon, a video, or a combination thereof.

The electrode module 120 may be provided in a cartridge housing 20 coupled to a handpiece 10. The electrode module 120 may include a plurality of first electrodes 121 of a needle type inserted into the skin S, and a plurality of second electrodes 122 of a contact type that contacts a surface of the skin S.

In this case, as shown in FIG. 3, the plurality of first electrodes 121 may be provided in a central portion of the cartridge housing 20, and the plurality of second electrodes 122 may be provided periphery of the plurality of first electrodes 121.

Here, an arrangement interval d1 of the plurality of first electrodes 121 may be provided to be wider than an arrangement interval d2 of the plurality of second electrodes 122. However, the present disclosure is not limited thereto, and the arrangement interval d1 of the plurality of first electrodes 121 may be provided to be narrower than the arrangement interval d2 of the plurality of second electrodes 121. At this time, the number of the plurality of first electrodes 121 and the number of the plurality of second electrodes 122 may be the same. The plurality of first electrodes 121 and the plurality of second electrodes 122 may generate abundant deep heat while concentrating the selected treatment area.

In addition, the number of the plurality of first electrodes 121 may be more or less than the number of the plurality of second electrodes 122. At this time, in the case that the arrangement interval d1 of the plurality of first electrodes 121 is wider than the arrangement interval d2 of the plurality of second electrodes 122, and the number of the plurality of first electrodes 121 is greater than the number of the plurality of second electrodes 122, relatively abundant deep heat may be generated more. And, in the case that the arrangement interval d1 of the plurality of first electrodes 121 is narrower than the arrangement interval d2 of the plurality of second electrodes 121, and the number of the plurality of first electrodes 121 is smaller than the number of the plurality of second electrodes 122, the selected treatment area may be relatively more concentrated.

The plurality of needle-type electrodes 121 may penetrate a deep portion of the skin S (e.g., the dermal layer) and remove damaged collagen, elastic fibers, and the like from the deep portion of a target point and promote new formation by using heat generated by at least one of a low-frequency current or a high-frequency current. The plurality of second electrodes 122 of the contact type may contact the stratum corneum, which is the surface of the skin S, and remove dead skin cells and promote new formation by using heat generated by at least one of the low-frequency current or the high-frequency current.

As illustrated in FIG. 5, among the plurality of first electrodes 121 of the needle type, a plurality of first electrodes 121a of plus (+) polarity may be arranged in the form of multiple groups in the electrode module 120. As illustrated in FIG. 6, among the plurality of second electrodes 122 of the contact type, a plurality of electrodes 122b of minus (−) polarity and a plurality of electrodes 122a of plus (+) polarity may be arranged in the form of multiple groups in the electrode module 120. For example, the plurality of second electrodes 122 of the contact type may be repeatedly arranged in the form of multiple groups of plus (+) polarity electrodes 122a and minus (−) polarity electrodes 122b on the upper side and right side of the cartridge housing 20, and the plurality of minus (−) polarity electrodes 122b and plus (+) polarity electrodes 122a on the lower side and left side of the cartridge housing 20.

The energy supply module 130 may apply a first current of a monopolar type to the plurality of first electrodes 121 and a second current of a bipolar type to the plurality of second electrodes 122, and may selectively apply the first current of the monopolar type to the plurality of first electrodes 121 and the second current of the bipolar type to the plurality of second electrodes 122. Here, the energy supply module 130 may include a first circuit for applying the first current of the monopolar type, a second circuit for applying the second current of the bipolar type, and a switching circuit for selectively applying the first current of the monopolar type and the second current of the bipolar type supplied through the first circuit and the second circuit.

In this case, as illustrated in FIG. 7, the energy supply module 130 may apply the first current of the monopolar type to the plurality of first electrodes 121a of plus (+) polarity and a return pad 123 of minus (−) polarity. In order for the high-frequency current to flow to the skin S, a current circuit needs to be formed. When the energy supply module 130 applies the first current of the monopolar type to the plurality of first electrodes 121a of plus (+) polarity and the return pad 122 of minus (−) polarity, high-frequency energy is concentrated on the plurality of electrodes 121a of plus (+) polarity, thereby enabling deeper heat transfer than the bipolar type, thereby generating abundant deep heat. Here, the return pad 123 may be a pad for grounding. At this time, the return pad 123 may be mounted on a part of the body or may be provided externally.

In addition, as illustrated in FIG. 8, the energy supply module 130 may apply the second current of the bipolar type to the plurality of second electrodes 122a of plus (+) polarity and the plurality of second electrodes 122b of minus (−) polarity among the plurality of second electrodes 122. When the energy supply module 130 applies the bipolar type second current to the plurality of second electrodes 122a of plus (+) polarity and the plurality of second electrodes 122b of minus (−) polarity, the current circuit is formed between the plurality of second electrodes 122a of plus (+) polarity and the plurality of second electrodes 122b of minus (−) polarity, so that excessive heat transfer may be prevented and a selected treatment area may be concentrated.

The transfer module 140 may move the electrode module 120 so that the plurality of first electrodes 121 reaches the target depth within the skin S and the plurality of second electrodes 122 contacts the surface of the skin S. The transfer module 140 may move the electrode module 120 in a vertical direction. The transfer module 140 may move the plurality of first electrodes 121 to reach the target depth within the skin S and may move the plurality of second electrodes 122 to contact the surface of the skin S.

The controller 150 may be implemented with a memory 151 that stores data on an algorithm for controlling the operation of components within the device or a program that reproduces the algorithm, and at least one processor 152 that performs the operation using the data stored in the memory 151. Here, the memory 151 and the processor 152 may be implemented as separate chips, respectively. In addition, the memory 151 and the processor 152 may also be implemented as a single chip.

The memory 151 may store data supporting various functions of the apparatus, a program for the operation of the controller, may store input/output data, and may store a plurality of application programs or applications run on the apparatus, data for the operation of the apparatus, and commands. At least some of these application programs may be downloaded from an external server via wireless communication.

The memory 151 may include at least one type of storage medium among a flash memory type, a hard disk type, an SSD (Solid State Disk type), an SDD (Silicon Disk Drive) type, a multimedia card micro type, a card type memory (for example, an SD or XD memory, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, or an optical disk. In addition, the memory 151 may be a database that is separate from the apparatus but connected by wire or wirelessly.

The memory 151 may store depth information for the skin layer, and mapping information that maps each treatment condition set to each depth of the skin layer.

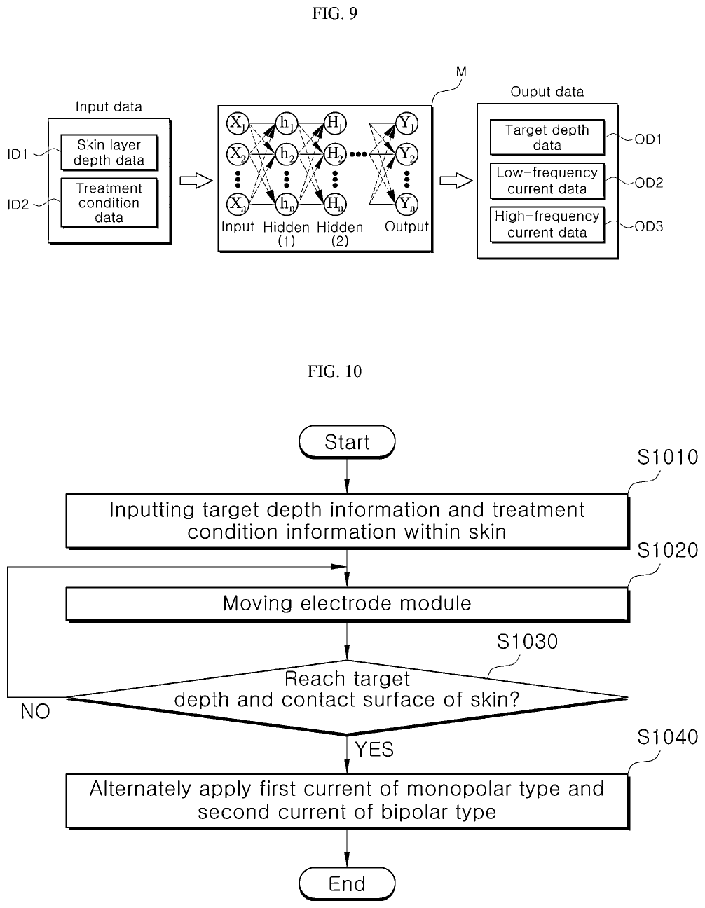

FIG. 9 is a diagram illustrating an example of a process of storing target depth data, low-frequency current data, and high-frequency current data in the memory of FIG. 1.

Referring to FIG. 9, a server 200 electrically connected to the controller 150 may learn the input values of skin layer depth data ID1 and treatment condition data ID2, which are metadata, based on a treatment recommendation model M, and output result values of target depth data OD1, low-frequency current data OD2, and high-frequency current data OD3 of the plurality of first electrodes 121 of the recommended needle type.

The treatment recommendation model M may be constructed to learn the skin layer depth data ID1 and the treatment condition data ID2 included in the input data through correlation. The treatment recommendation model M may be constructed and reinforced learning as a learning data set using a CNN algorithm or an RNN algorithm for the depth of the skin layer and treatment conditions.

The server 200 may transmit the target depth data OD1, the low-frequency current data OD2, and the high-frequency current data OD3 of the plurality of first electrodes 121 determined based on the target depth information and the treatment condition information within the skin input through the input module 110 to the memory 151 of the controller 150. The memory 151 may store the received target depth data OD1, the low-frequency current data OD2, and the high-frequency current data OD3 of the plurality of first electrodes 121.

Here, the depth data ID1 of the skin layer may be a depth value within the dermis layer, and the treatment condition data ID2 may be data for each treatment condition, for example, data for removing wrinkles, data for restoring skin elasticity, data for removing sebum, and the like.

The processor 152 may control the energy supply module 130 so that when the plurality of first electrodes 121 reaches the target depth of the skin layer and the plurality of second electrodes 122 contacts the surface of the skin, the first current of the monopolar type is alternately applied to the plurality of first electrodes 121 and the second current of the bipolar type is alternately applied to the plurality of second electrodes 122.

The processor 152 may control the energy supply module 130 so that the first current of the monopolar type is applied to the plurality of first electrodes 121 with a first current intensity corresponding to the reached target depth, and the second current of the bipolar type is applied to the plurality of second electrodes 122 with a second current intensity corresponding to the surface of the contacted skin. Here, the first current intensity and the second current intensity may be the same as or different from each other, and the first current intensity and the second current intensity may be at least one of a low-frequency current or a high-frequency current. In addition, as the target depth becomes deeper, the first current intensity of the monopolar type may be greater or less than the second current intensity of the bipolar type.

The processor 152 may control the energy supply module 130 so that, when the plurality of first electrodes 121 reaches the target depth of the skin layer and the plurality of second electrodes 122 contacts the surface of the skin, the first current of the monopolar type and the second current of the bipolar type are applied in a preset order of low-frequency current and high-frequency current for a preset time. The plurality of first electrodes 121 may sequentially change the first current of the monopolar type corresponding to the treatment condition for the target depth and apply it from the low-frequency current to the high-frequency current, and the plurality of second electrodes 122 may sequentially change the second current of the bipolar type from the low-frequency current to the high-frequency current and apply it to the surface of the skin. At this time, the plurality of first electrodes 121 and the plurality of second electrodes 122 may continuously increase the first current of the monopolar type and the second current of the bipolar type from the low frequency current to the high frequency current and apply them, and may continuously increase the first current of the monopolar type and the second current of the bipolar type from the low frequency current to the high frequency current while having a pause period for a preset unit time.

The processor 152 may control the energy supply module 130 so that, when the plurality of first electrodes 121 reaches the target depth of the skin layer, the first current of the monopolar type corresponding to the corresponding treatment condition for the target depth is applied as one of the corresponding low frequency current and the corresponding high frequency current for each preset target depth for a preset time based on the mapping information. The processor 152 may control the energy supply module 130 to apply the first current of the monopolar type as one of the corresponding low-frequency current and corresponding high-frequency current for each target depth based on the target depth data OD1, the low-frequency current data OD2, and the high-frequency current data OD3 of the plurality of first electrodes 121 stored in the memory 151. The plurality of first electrodes 121 may apply the first current of the monopolar type as one of the corresponding low-frequency current and corresponding high-frequency current for each target depth.

The processor 152 may measure skin impedance based on the plurality of first electrodes 121 or the plurality of second electrodes 122, and output energy corresponding to the measured skin impedance among the preset energies. At this time, the processor 152 may further control the energy supply module 130 so that the plurality of second electrodes 122 transmits energy corresponding to the skin impedance to the skin surface, and the plurality of first electrodes 121 transmits energy corresponding to the skin impedance when the target depth is reached.

For example, the processor 152 may measure first skin impedance based on the plurality of first electrodes 121 and output energy corresponding to the measured first skin impedance among the preset energies. At this time, the processor 152 may further control the energy supply module 130 so that the plurality of second electrodes 122 transmits energy corresponding to the first skin impedance to the skin surface, and the plurality of first electrodes 121 transmits energy corresponding to the first skin impedance when the target depth is reached.

For another example, the processor 152 may measure second skin impedance based on the plurality of second electrodes 122 and output energy corresponding to the measured second skin impedance among the preset energies. At this time, the processor 152 may further control the energy supply module 130 so that the plurality of second electrodes 122 transmits energy corresponding to the second skin impedance to the skin surface and the plurality of first electrodes 121 transmit energy corresponding to the second skin impedance when the target depth is reached.

For another example, the processor 152 may measure the first skin impedance based on the plurality of first electrodes 121, measure the second skin impedance based on the plurality of second electrodes 122, and output energy corresponding to the measured first skin impedance and energy corresponding to the measured second skin impedance among the preset energies. At this time, the processor 152 may further control the energy supply module 130 so that the plurality of second electrodes 122 transfers energy corresponding to the first skin impedance to the skin surface, and the plurality of first electrodes 121 transfers energy corresponding to the second skin impedance when the target depth is reached. In addition, the processor 152 may further control the energy supply module 130 so that the plurality of second electrodes 122 transfers energy corresponding to the second skin impedance to the skin surface, and the plurality of first electrodes 121 transfers energy corresponding to the first skin impedance when the target depth is reached.

FIG. 10 is a flowchart showing a method for supplying monopolar and bipolar current by skin contact and skin depth according to the present disclosure.

Referring to FIG. 10, the method for supplying monopolar and bipolar current by skin contact and skin depth may include an input step S1010, a movement step S1020, a determination step S1030, and a control step S1040.

The input step may receive target depth information and treatment condition information within the skin from the user through the input unit 110 (step S1010). Here, the target depth information within the skin may be a target depth value within the dermis layer, and the treatment condition information may be a treatment condition for removing wrinkles, a treatment condition for restoring skin elasticity, a treatment condition for removing sebum, and the like.

The movement step may move the electrode module 120 through the transfer module 140 so that the plurality of first electrodes 121 reaches the target depth within the skin S and the plurality of second electrodes 122 contacts the surface of the skin S (step S1020). The processor 152 may control the transfer module 140 to move the electrode module 120 so that the plurality of first electrodes 121 reaches the target depth within the skin S based on the target depth information and treatment condition information within the skin S input through the input module 110. The plurality of first electrodes 121 may penetrate to each target depth within the dermis layer corresponding to the corresponding condition among the treatment conditions for removing wrinkles, the treatment conditions for restoring skin elasticity, and the treatment conditions for removing sebum. The plurality of second electrodes 122 may contact the surface of the skin S to remove keratin in the stratum corneum.

The determination step may determine whether the plurality of first electrodes 121 reaches the target depth of the skin layer and the plurality of second electrodes 122 contacts the surface of the skin through the processor 152 (step S1030). For example, the processor 152 may determine whether the plurality of first electrodes 121 reaches each target depth in the dermis layer corresponding to the corresponding condition among the treatment condition for removing wrinkles, the treatment condition for restoring skin elasticity, and the treatment condition for removing sebum. In addition, the processor 152 may determine whether the plurality of second electrodes 122 contacts the surface of the skin S to remove dead skin cells of the stratum corneum.

The control step may control the energy supply module 130 to alternately apply the first current of the monopolar type to the plurality of first electrodes 121 and the second current of the bipolar type to the plurality of second electrodes 122 in the case that the plurality of first electrodes 121 reaches the target depth of the skin layer and the plurality of second electrodes 122 contacts the surface of the skin (step S1040). The processor 152 may control the energy supply module 130 to alternately apply the first current of the monopolar type to the plurality of first electrodes 121 at the first current intensity corresponding to the reached target depth and apply the second current of the bipolar type to the plurality of second electrodes 122 at the second current intensity corresponding to the surface of the contacted skin. Here, the first current intensity and the second current intensity may be the same or different from each other, and the first current intensity and the second current intensity may be at least one of a low-frequency current and a high-frequency current. In addition, as the target depth becomes deeper, the first current intensity of the monopolar type may be greater or less than the second current intensity of the bipolar type.

The control step may further control the energy supply module 130 through the processor 152 to measure skin impedance based on the plurality of first electrodes 121 or the plurality of second electrodes 122, output energy corresponding to the measured skin impedance among preset energies, and the plurality of second electrodes 122 may transmit energy corresponding to the skin impedance to the skin surface, and the plurality of first electrodes 121 may transmit energy corresponding to the skin impedance when the target depth is reached.

For example, the processor 152 may measure the first skin impedance based on the plurality of first electrodes 121 and output energy corresponding to the measured first skin impedance among the preset energies. At this time, the processor 152 may further control the energy supply module 130 so that the plurality of second electrodes 122 transmits energy corresponding to the first skin impedance to the skin surface, and the plurality of first electrodes 121 transmits energy corresponding to the first skin impedance when the target depth is reached.

For another example, the processor 152 may measure the second skin impedance based on the plurality of second electrodes 122 and output energy corresponding to the measured second skin impedance among the preset energies. At this time, the processor 152 may further control the energy supply module 130 so that the plurality of second electrodes 122 transmits energy corresponding to the second skin impedance to the skin surface, and the plurality of first electrodes 121 transmits energy corresponding to the second skin impedance when the target depth is reached.

As another example, the processor 152 may measure the first skin impedance based on the plurality of first electrodes 121, measure the second skin impedance based on the plurality of second electrodes 122, and output the energy corresponding to the measured first skin impedance and the energy corresponding to the measured second skin impedance among the preset energies. At this time, the processor 152 may further control the energy supply module 130 so that the plurality of second electrodes 122 transmits energy corresponding to the first skin impedance to the skin surface, and the plurality of first electrodes 121 transmits energy corresponding to the second skin impedance when the target depth is reached. In addition, the processor 152 may further control the energy supply module 130 so that the plurality of second electrodes 122 transmits energy corresponding to the second skin impedance to the skin surface, and the plurality of first electrodes 121 transmits energy corresponding to the first skin impedance when the target depth is reached.

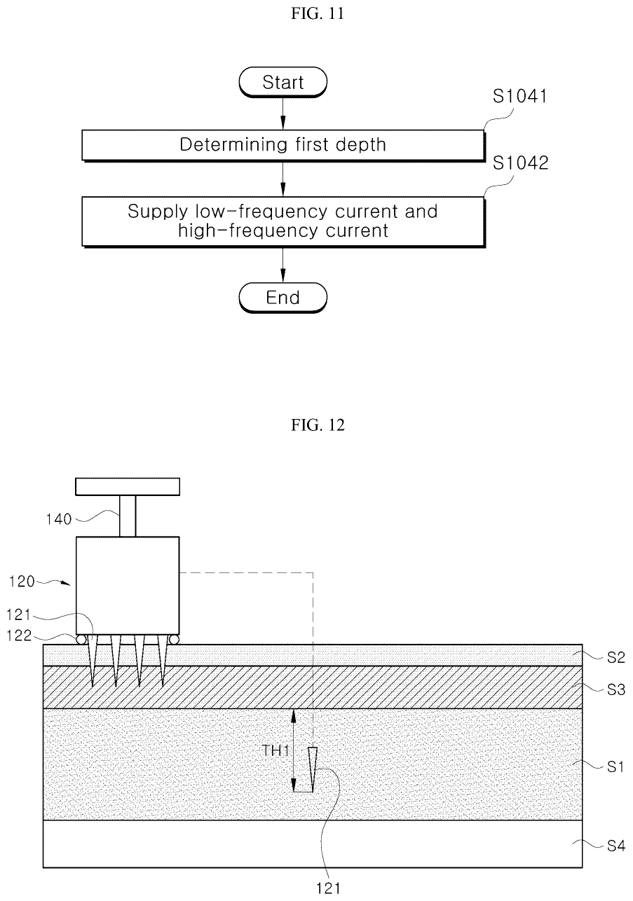

FIGS. 11 and 12 are diagrams illustrating an example of a process of controlling low-frequency current and high-frequency current to be sequentially applied to the plurality of first electrodes in the control step of FIG. 10.

Referring to FIGS. 11 and 12, the control step may determine whether the target depth is a preset first depth TH 1 through the processor 152 (step S1041). The processor 152 may control the energy supply module 130 so that the first current of the monopolar type and the second current of the bipolar type are applied in the order of applying the preset low-frequency current and high-frequency current for a preset time until the target depth reaches the first depth TH1 (step S1042). The plurality of first electrodes 121 may sequentially change the first current of the monopolar type corresponding to the corresponding treatment condition from the low-frequency current to the high-frequency current and apply it until the first depth TH1 is reached, and the plurality of second electrodes 122 may sequentially change the second current of the bipolar type from the low-frequency current to the high-frequency current and apply it to the surface of the skin. At this time, the plurality of first electrodes 121 and the plurality of second electrodes 122 may continuously increase the first current of the monopolar type and the second current of the bipolar type from the low frequency current to the high frequency current and apply them, and may continuously increase the first current of the monopolar type and the second current of the bipolar type from the low frequency current to the high frequency current while having a pause section for a preset unit time. At this time, S2 may be the stratum corneum, S3 may be the epidermal layer, and S4 may be the subcutaneous tissue layer.

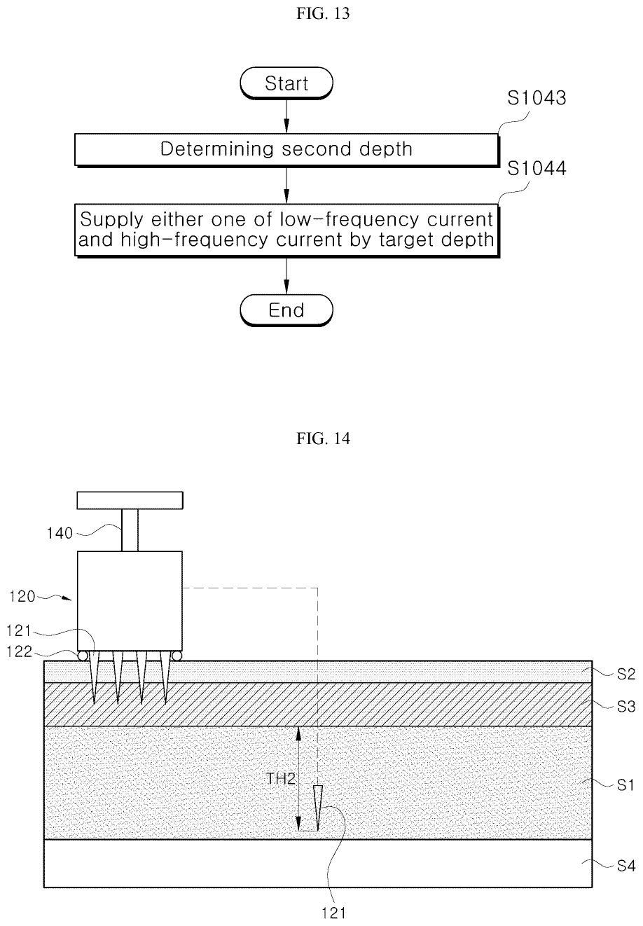

FIGS. 13 and 14 are diagrams illustrating an example of a process of controlling the application of the corresponding low frequency current and the corresponding high frequency current to the plurality of first electrodes according to the target depth in the control step of FIG. 10.

Referring to FIGS. 13 and 14, the control step may determine whether the target depth is a preset second depth TH 2 through the processor 152 (step S1043). In the case that the target depth is the second depth TH2, the processor 152 may control the energy supply module 130 so that the first current of the monopolar type corresponding to the treatment condition for the second depth TH2 is applied as one of the corresponding low-frequency current and the corresponding high-frequency current for each preset second depth for a preset time based on the mapping information (step S1044). The processor 152 may control the energy supply module 130 so that the first current of the monopolar type is applied as one of the corresponding low-frequency current and the corresponding high-frequency current for each second depth based on the target depth data OD1, the low-frequency current data OD2, and the high-frequency current data OD3 of the plurality of first electrodes 121 stored in the memory 151. The plurality of first electrodes 121 may apply the first current of the monopolar type as at least one of a corresponding low-frequency current and a corresponding high-frequency current for each second depth.

Meanwhile, a computer program stored in a computer-readable storage medium according to another aspect of the present disclosure may perform an operation of moving the electrode module of the apparatus through the transfer module of the apparatus so that the first electrode reaches a target depth within the skin and the second electrode contacts the surface of the skin.

Thereafter, the computer program stored in the computer-readable storage medium may perform an operation of determining, through the controller of the apparatus, whether the first electrode reaches the target depth and the second electrode contacts the surface of the skin.

Later, the computer program stored in the computer-readable storage medium may perform an operation of controlling the energy supply module of the apparatus so that the first current and the second current are alternately applied when the first electrode reaches the target depth and the second electrode contacts the surface of the skin through the controller of the apparatus. At this time, the first electrode and the second electrode are provided in the cartridge housing coupled to the handpiece, and the first electrode may be provided in the central portion of the cartridge housing, and the second electrode may be provided in the periphery of the first electrode.

At this time, the control operation may further control the energy supply module so that the skin impedance is measured based on the first electrode or the second electrode, energy corresponding to the measured skin impedance among the preset energies is output, the second electrode transmits energy corresponding to the skin impedance to the skin surface, and the first electrode transmits energy corresponding to the skin impedance when the target depth is reached.

In addition, the control operation may control the energy supply module so that the first current is applied to the first electrode with the first current intensity corresponding to the reached target depth, and the second current is applied to the second electrode with the second current intensity corresponding to the surface of the contacted skin, through the controller, whereby the first current intensity and the second current intensity may be the same as or different from each other. At this time, the first current intensity and the second current intensity may be at least one of a low-frequency current or a high-frequency current.

In addition, the control operation may control the energy supply module so that, when the first electrode reaches the target depth and the second electrode contacts the surface of the skin, the first current and the second current are applied in the order of application of the preset low-frequency current and the preset high-frequency current for a preset time through the controller.

In addition, the control operation may control the energy supply module so that, when the first electrode reaches the target depth, the first current is applied as one of the corresponding low-frequency current and the corresponding high-frequency current for each preset target depth for a preset time through the controller.

At least one component may be added or deleted in accordance with the performance of the components illustrated in FIGS. 1 to 9. In addition, it will be readily understood by those skilled in the art that the mutual positions of the components may be changed in accordance with the performance or structure of the system.

Although FIG. 10, FIG. 11, and FIG. 13 describe the execution of multiple steps sequentially, this is only an exemplary description of the technical idea of the present embodiment, and a person having ordinary knowledge in the technical field to which the present embodiment belongs may modify and change the order described in FIG. 10, FIG. 11, and FIG. 13 without departing from the essential features of the present embodiment, or may modify and change one or more of the multiple steps in parallel, and thus FIG. 10, FIG. 11, and FIG. 13 are not limited to a sequential order.

As described above, the disclosed embodiments have been described with reference to the attached drawings. A person having ordinary knowledge in the technical field to which the present disclosure belongs will understand that the present disclosure may be implemented in a form different from the disclosed embodiments without changing the technical idea or essential features of the present disclosure. The disclosed embodiments are exemplary and should not be construed as limiting.

1. An apparatus for supplying monopolar and bipolar current by skin contact and skin depth, comprising:
an electrode module including a plurality of first electrodes of a needle type inserted into a skin and a plurality of second electrodes of a contact type contacting a surface of the skin;
an energy supply module configured to apply a first current of a monopolar type to the first electrode and a second current of a bipolar type to the second electrode;
a transfer module configured to move the electrode module so that the first electrode reaches a target depth within the skin and the second electrode contacts the surface of the skin; and
a controller configured to control the energy supply module so that the first current and the second current are alternately applied based on the first electrode reaching the target depth and the second electrode contacting the surface of the skin,
wherein the first electrode and the second electrode are provided in a cartridge housing coupled to a handpiece, and
wherein the first electrode is provided in a central portion of the cartridge housing, and the second electrode is provided in a periphery of the first electrode.

2. The apparatus according to claim 1, wherein the controller is configured to:
measure skin impedance based on the first electrode or the second electrode, and output energy corresponding to the measured skin impedance among preset energies, and
further control the energy supply module so that the second electrode transmits energy corresponding to the skin impedance to the skin surface, and the first electrode transmits energy corresponding to the skin impedance when the target depth is reached.

3. The apparatus according to claim 1, wherein the controller is configured to:
control the energy supply module so that the first current is applied to the first electrode at a first current intensity corresponding to the reached target depth, and the second current is applied to the second electrode at a second current intensity corresponding to the surface of the contacted skin,
wherein the first current intensity and the second current intensity are identical or different from each other.

4. The apparatus according to claim 1, wherein the first current intensity and the second current intensity are at least one of a low-frequency current or a high-frequency current.

5. The apparatus according to claim 4, wherein the controller is configured to:
control the energy supply module so that, based on the first electrode reaching the target depth and the second electrode contacting the surface of the skin, the first current and the second current are applied in a preset application order of the low-frequency current and the high-frequency current for a preset time.

6. The apparatus according to claim 4, wherein the controller is configured to:
control the energy supply module so that, based on the first electrode reaching the target depth, the first current is applied to either one of the low-frequency current and the high-frequency current for a preset time and for a preset target depth.

7. A method performed by an apparatus for supplying monopolar and bipolar current by skin contact and skin depth, the method comprising:
moving, by a transfer module of the apparatus, an electrode module of the apparatus so that a first electrode reaches a target depth within the skin and a second electrode contacts a surface of the skin;
determining, by a controller of the apparatus, whether the first electrode reaches the target depth and the second electrode contacts the surface of the skin; and
controlling, by the controller of the apparatus, an energy supply module of the apparatus so that the first current and the second current are alternately applied based on the first electrode reaching the target depth and the second electrode contacting the surface of the skin,
wherein the first electrode and the second electrode are provided in a cartridge housing coupled to a handpiece, and
wherein the first electrode is provided in a central portion of the cartridge housing, and the second electrode is provided in a periphery of the first electrode.

8. The method according to claim 7, wherein controlling the energy supply module of the apparatus includes:
measuring skin impedance based on the first electrode or the second electrode, and outputting energy corresponding to the measured skin impedance among preset energies, and
controlling the energy supply module so that the second electrode transmits energy corresponding to the skin impedance to the skin surface, and the first electrode transmits energy corresponding to the skin impedance when the target depth is reached.

9. The method according to claim 7, wherein controlling the energy supply module of the apparatus includes:
controlling, by the controller, the energy supply module so that the first current is applied to the first electrode at a first current intensity corresponding to the reached target depth, and the second current is applied to the second electrode at a second current intensity corresponding to the surface of the contacted skin,
wherein the first current intensity and the second current intensity are identical or different from each other.

10. The method according to claim 9, wherein the first current intensity and the second current intensity are at least one of a low-frequency current or a high-frequency current.

11. The method according to claim 10, wherein controlling the energy supply module of the apparatus includes:
controlling, by the controller, the energy supply module so that, based on the first electrode reaching the target depth and the second electrode contacting the surface of the skin, the first current and the second current are applied in a preset application order of the low-frequency current and the high-frequency current for a preset time.

12. The method according to claim 10, wherein controlling the energy supply module of the apparatus includes:
controlling, by the controller, the energy supply module so that, based on the first electrode reaching the target depth, the first current is applied to either one of the low-frequency current and the high-frequency current for a preset time and for a preset target depth.
